(19) United States
(12) Patent Application Publication
(10) Pub. No.: US 2021/0361796 A1
(43) Pub. Date: Nov. 25, 2021

(54) SANITIZING SYSTEM FOR SANITIZING DEVICES AND METHOD THEREFOR

(71) Applicant: Mark Innovation LLC, Mattapoisett, MA (US)

(72) Inventors: Jacob MARK, Needham, MA (US); Joe MARK, Somerville, MA (US)

(21) Appl. No.: 17/326,431

(22) Filed: May 21, 2021

Related U.S. Application Data
(60) Provisional application No. 63/029,139, filed on May 22, 2020.

(51) Int. Cl. A61L 2/10

(52) U.S. Cl. CPC A61L 2/10; A61L 2202/14; A61L 2202/122; A61L 2202/16

(57) ABSTRACT

A sanitizing system for sanitizing multiple shared fungible handheld devices, the system including a sanitizing station including a closure configured to hold enclosed therein at least one of the multiple shared fungible handheld devices, and a radiation source of UV-C radiation coupled to the closure and disposed to irradiate surface(s) of the at least one of multiple shared fungible handheld devices held in the closure effecting sanitization of unsanitary surface(s) of the at least one of the multiple shared fungible handheld devices to a predetermined sanitization characteristic that determines the surface(s) being sanitary, and a sensor configured so as to measure a dosage of radiation received by the unsanitary surface(s) of the at least one of multiple shared fungible handheld devices, via the radiation source, and signal receipt of efficacious measured dose of radiation sanitization to each of the at least one of the multiple shared fungible handheld devices.

### CROSS-REFERENCE TO RELATED APPLICATIONS

This application is a non-provisional which claims priority from and the benefit of United States Provisional Patent Application No. 63/029,139, filed on May 22, 2020, the disclosure of which is incorporated by reference herein in its entirety.

### BACKGROUND

### 1. Field

The exemplary embodiment generally relates to sanitizing systems, more particularly, to sanitizing systems for sanitizing multiple shared fungible devices communicably connected to a communication network.

### 2. Brief Description of Related Developments

In a typical commercial or enterprise environment, (e.g., retail shopping store) users push carts or carry baskets through any number of aisles, adding items to their carts or baskets from the many shelves and/or displays in accordance with, e.g., a prepared shopping list, in-store advertising, mere fancy, etc. Users spend most of their shopping time negotiating the many aisles, locating various items for purchase, and waiting to checkout at the register. As a result, some stores have implemented shared handheld or wearable devices for users to utilize, scanning items while the user shops. The shared handheld or wearable devices help to reduce a user's time spent within the store, as scanning items while shopping eliminates the need to wait for a cashier to scan the items at the register (i.e., upon completion of picking the items, the user merely pays and leaves).

Other enterprise environments include, e.g., hospitals, commercial offices, etc., where shared fungible handheld or wearable devices (e.g., mobile phones, tablets, or personal digital assistants (PDAs), etc.), that allow users to interface with the enterprise's systems roaming the facilities, may also be shared/exchanged across multiple users.

### BRIEF DESCRIPTION OF THE DRAWINGS

The foregoing aspects and other features of the present disclosed embodiment are explained in the following description, taken in connection with the accompanying drawings, wherein:

FIG. 1 is a schematic block diagram of a system for sanitizing devices in accordance with the aspects of the disclosed embodiment;

FIG. 1A is a schematic block diagram of a system for sanitizing devices in accordance with the aspects of the disclosed embodiment;

FIGS. 2A-2C are schematic illustrations of various devices in accordance with the aspects of the disclosed embodiment;

FIGS. 3A-3C are schematic illustrations of portions of the system of FIG. 1 in accordance with the aspects of the disclosed embodiment;

FIGS. 4A-4C are schematic illustrations of portions of the system of FIG. 1 in accordance with the aspects of the disclosed embodiment;

FIGS. 5A-5C are schematic illustrations of portions of the system of FIG. 1 in accordance with the aspects of the disclosed embodiment;

FIGS. 6A-6C are schematic illustrations of portions of the system of FIG. 1 in accordance with the aspects of the disclosed embodiment;

FIG. 7 is a schematic block diagram of a portion of the system of FIG. 1 in accordance with the aspects of the disclosed embodiment;

FIG. 8 is a block diagram illustrating an exemplary method of sanitizing a device;

FIG. 9 is a block diagram illustrating an exemplary method of sanitizing a device;

FIG. 10 is a schematic illustration of portions of the system of FIG. 1A in accordance with the aspects of the disclosed embodiment;

FIG. 11 is a schematic illustration of portions of the system of FIG. 1A in accordance with the aspects of the disclosed embodiment; and

FIG. 12 is a block diagram illustrating an exemplary method of sanitizing a device.

### DETAILED DESCRIPTION

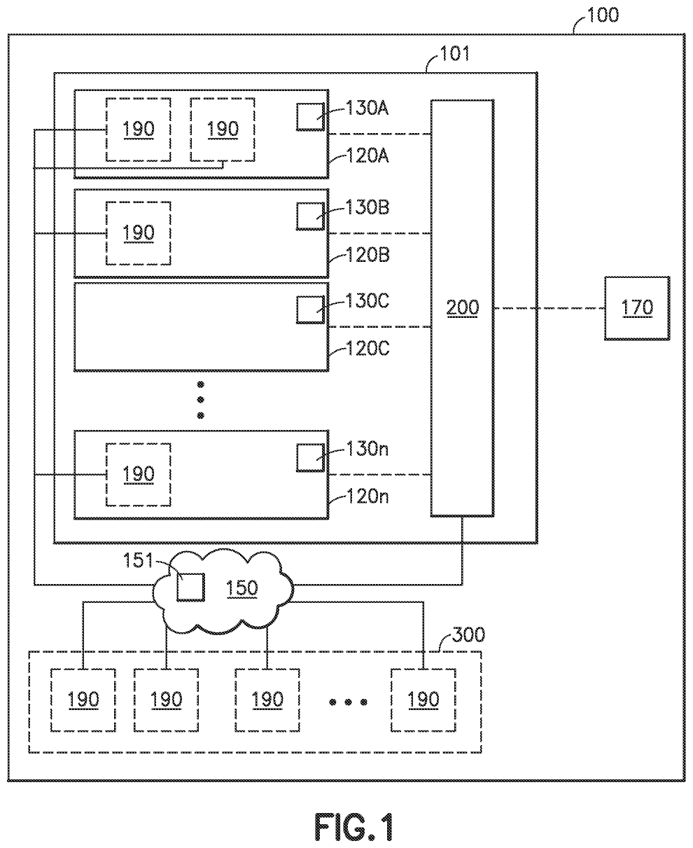

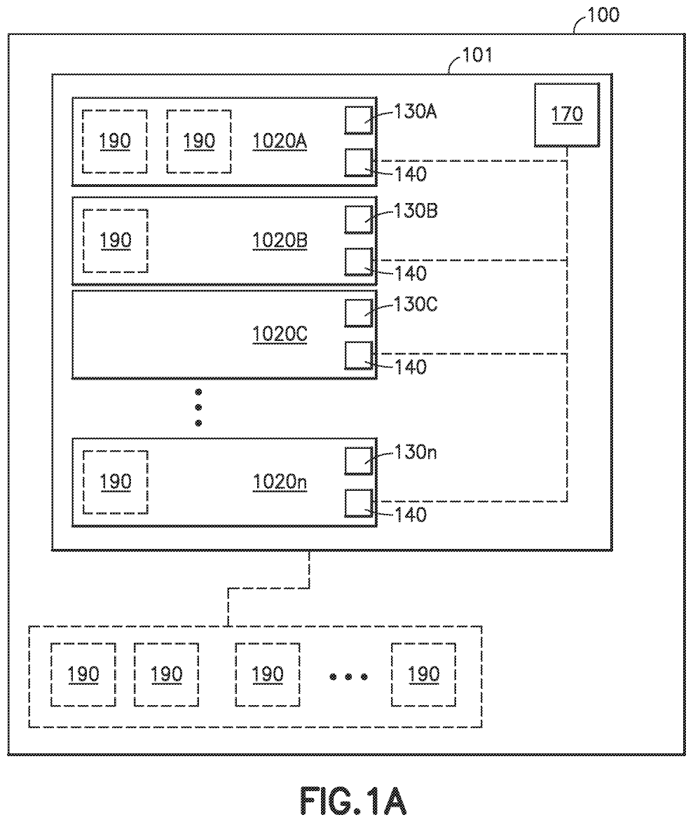

FIG. 1 illustrates an exemplary schematic diagram of a sanitizing system 100 for sanitizing one or more shared fungible handheld or wearable devices 190. The sanitizing system 100 has a sanitizing station(s) 101 for sanitizing the shared fungible handheld or wearable device(s) 190 communicably connected to the sanitizing station(s) 101 via a communication network 150. Although the aspects of the disclosed embodiment will be described with reference to the drawings, it should be understood that the aspects of the disclosed embodiment can be embodied in many alternate forms. In addition, any suitable size, shape or type of elements or materials could be used.

Generally, with systems having multiple shared fungible (i.e., one device may be interchangeable with any other device) handheld or wearable devices 190 utilized by many users and are fomites, it is desirable to sanitize the shared fungible handheld or wearable device(s) 190 (fomites) between uses and confirm the shared fungible handheld or wearable device(s) 190 is sanitized for each user's use. The shared fungible handheld or wearable device(s) 190 are typically highly germ-laden due to repeated exposure to multiple different users. The repeated exposure to multiple different users creates a breeding ground for, e.g., bacteria and viruses. An example of a system with shared fungible handheld or wearable device(s) 190 is in the context of retail locations/stores (such as, e.g., the system described in U.S. Pat. No. 7,856,368, published Dec. 21, 2010, the disclosure of which is incorporated herein in its entirety); while in other aspects, multiple fungible shared handheld or wearable devices are used in the context of a hospital or healthcare facilities (an example which is described in U.S. Pat. No. 9,904,777, published Feb. 27, 2018, the disclosure of which is incorporated herein in its entirety), or any other suitable context. The sanitizing system 100 for sanitizing the shared fungible handheld or wearable device(s) 190 may be utilized to sanitize surfaces of the shared fungible handheld or wearable device(s) 190 of pathogens, e.g., bacteria and viruses located thereon.

Still referring to FIG. 1, the sanitizing system 100 includes the sanitizing station(s) 101 which is located, for example, in a waiting room of a hospital or healthcare facility for healthcare worker or patient use (e.g., the shared fungible handheld or wearable device(s) 190 may be utilized between patients for checking in and sanitized at the sanitizing station(s) 101 between each use) or located at the entrance/exit to a retail store for shoppers use (e.g., the shared fungible handheld or wearable device(s) 190 may be utilized between shoppers for scanning items and sanitized at the sanitizing station(s) 101 between each use). The sanitizing station(s) 101 having at least one closure 120A-n configured to hold, enclosed therein, one or more of the shared fungible handheld or wearable device(s) 190 and a radiation source 130A-n (e.g., a UV lamp/tube, UV led, etc.). The closure(s) 120A-n may be located in proximity to each other in a common location, or may be distributed at different locations (collectively referred to as a sanitizing station or stations). The sanitizing system 100 effects sanitizing via dynamic dosage (i.e., dynamic determination of dosage effects sanitization), thus is more efficient (less power consumption, faster sanitization cycle times enabled) with higher confidence of sanitization that conventional systems The sanitizing system 100 further includes a transceiver 200 connected, at least, to the sanitizing station(s) 101. In one aspect, the transceiver 200 is configured to communicate via a communication link through the communication network 150, with at least one of the shared fungible handheld or wearable device(s) 190 within a respective closure 120A-n. In one aspect, the transceiver 200 is configured to communicate with at least one of the shared fungible handheld or wearable device(s) 190 in any suitable manner, such as, Bluetooth, as will be described below. In one aspect, the sanitizing station(s) 101 further includes a controller 170 configured to control various aspects of the sanitizing system 100 as will be described in detail herein. The controller 170 may be programmed to include a device accountant 172.

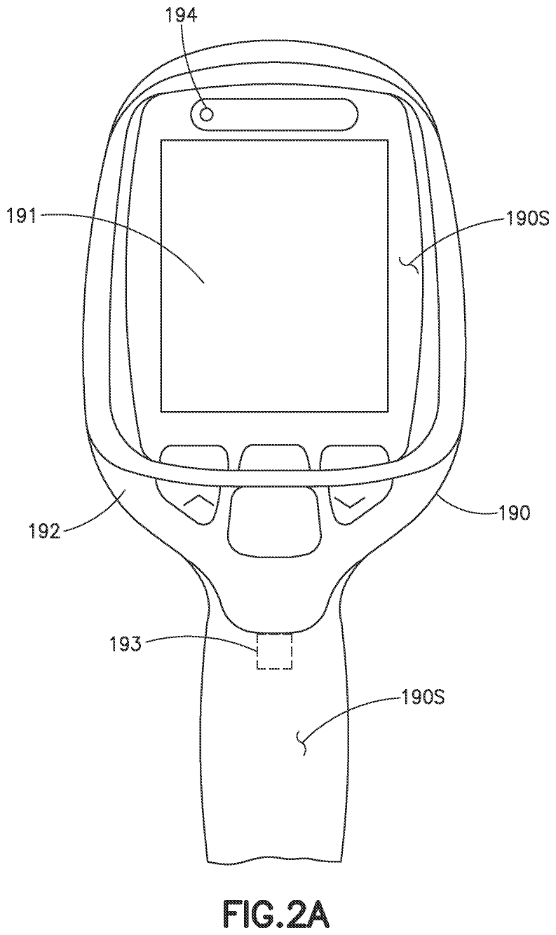

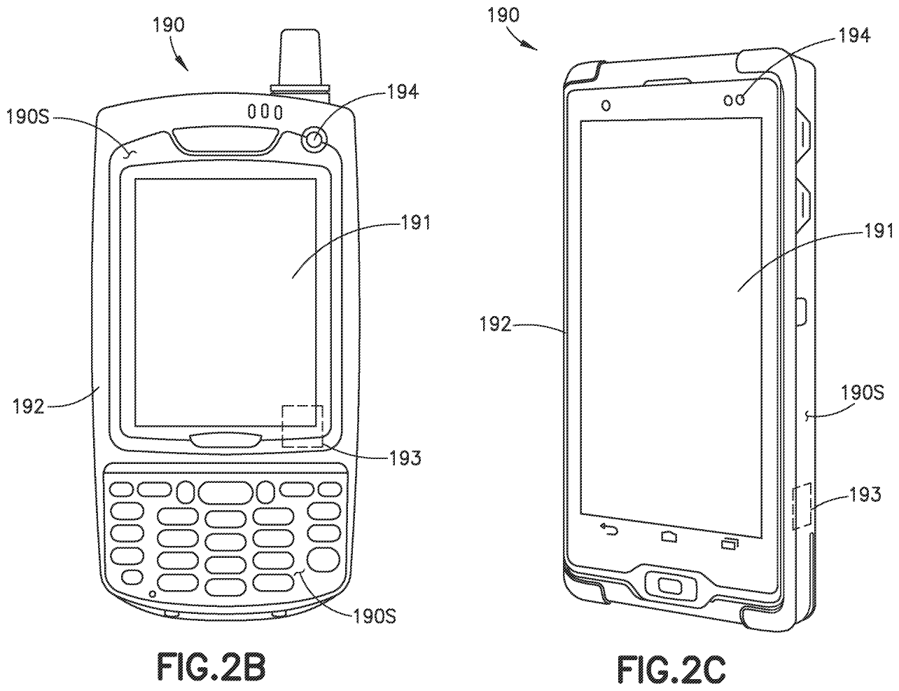

As will be described below, each closure 120A-n of the sanitizing station(s) 101, with a respective radiation source 130A-n disposed therein (i.e., coupled to an interior surface 123S of the closure 120A-n), is configured so as to irradiate surface(s) 190S (FIG. 2) of a respective one of the shared fungible handheld or wearable device(s) 190 held within the respective closure 120A-n. Irradiating the surface(s) 190S of the respective one of the shared fungible handheld or wearable device(s) 190 effects sanitization of unsanitary surface(s) to a predetermined sanitization characteristic (e.g., a characteristic determined by a predetermined dosage (e.g., between about 40 mW/cm² and about 50 mW/cm², but may be less than 40 mW/cm² or more than 50 mW/cm²) of UV-C radiation effecting a determined disruption to potential pathogens), or any other suitable characteristic). In one aspect, the transceiver 200 communicates, via the communication network 150, an indication from the sanitizing station(s) 101 of the surface(s) 190S of the respective one of the shared fungible handheld or wearable device(s) 190 in the respective closure 120A-n are sanitary. In one aspect, an indication may be a local indication (without transceiver communication), as described below, or received (via transceiver) by the respective one of the shared fungible handheld or wearable device(s) 190 and displayed on a display (e.g., a graphical user interface, lights, or any other visual indicator) of each respective one of the shared fungible handheld or wearable device(s) 190 to confirm the surface(s) 190S of the respective shared fungible handheld or wearable device(s) 190 being in a sanitized state. In one aspect, the communication to the respective shared fungible handheld or wearable device(s) 190 from the transceiver 200 allows a user to be able to determine which of the shared fungible handheld or wearable device(s) 190 are in a confirmed sanitized state and ready for utilization within the retail location (i.e., a change in appearance of the display screen 191 of a respective one of the shared fungible handheld or wearable device(s) 190 or any other suitable change in appearance may form the indication). In one aspect, the individual docking stations 30 of a respective one of the shared fungible handheld or wearable device(s) 190 may include an indicator, such as, e.g., a LED light, to indicate when the respective one of the shared fungible handheld or wearable device(s) 190 is sanitary from a signal of a dosimetry sensor as described below.

Referring to FIGS. 1 and 2A-2C, in one aspect, the shared fungible handheld device(s) 190 may be one or more portable devices having a screen-based display user interface, where the portable devices are used in, e.g., a hospital, healthcare facility, or retail environment/location. Although the aspects of the disclosed embodiment will be described herein with respect to handheld devices within a hospital, healthcare facility, or retail environment/location, the disclosed embodiment may be utilized with any portable handheld or wearable devices, such as, Personal Digital Assistants (PDAs), computer terminals, cellular phones, tablets, or any other suitable devices, in any suitable commercial (or non-commercial) environment where multiple fungible handheld devices are shared between multiple users.

In this aspect, the shared fungible handheld device(s) 190 are utilized by multiple different users for, e.g., shopping in a retail store, or gather data, track workload, and document patient outcomes within a healthcare facility and are configured for receiving, storing, and/or displaying, e.g., in the context of retail, a personalized shopping list for the retail store, items being purchased in the retail store, or in the context of healthcare, patient data, etc. Each of the shared fungible handheld device(s) 190 is portable, e.g., carried in the palm of or held by a user's hand while roaming the facilities. The structure of the shared fungible handheld device(s) 190 generally comprises a display screen or panel 191, and a display screen housing 192. Enclosed in the housing are components 193, such as, e.g., a transceiver, an antenna, a power source, a microprocessor for operating the shared fungible handheld device(s) 190, and short-term, i.e., RAM, and long-term, i.e., ROM, memory storage. Additional structure of the shared fungible handheld device(s) 190 may include a reader for reading, e.g., loyalty cards or bar code from a patient wrist band, a two-way speaker/microphone system and the circuitry and electronics therefor.

The display screen 191 of the shared fungible handheld device(s) 190 may be a touch sensitive, liquid crystal display (“LCD”) panel. The LCD panel may be protected by an overlying panel protection layer to protect the display screen from damage, such as, from water or other liquids, scratches, dirt, dust, etc. The display screen 191 may be configured so as to display the indication of the surface(s) 190S of the shared fungible handheld device(s) 190 being sanitary as will be described in greater detail below. In other aspects, the shared fungible handheld device(s) 190 include any suitable visual and/or aural indication of being sanitary. For example, the shared fungible handheld device(s) 190 may include any other suitable indicator such as an LED light 194 which illuminates to indicate the surface(s) 190S of the respective one of the shared fungible handheld device(s) 190 are sanitary. Audible sounds emitted from a speaker of the respective one of the shared fungible handheld device(s) 190 may also be provided. The indication received by the shared fungible handheld device(s) 190 is displayed by the shared fungible handheld device(s) 190 so as to present confirmation of sanitization of the shared fungible handheld device(s) 190 as will be further described below. The displaying of the indication provides a user with confirmation of sanitization of the shared fungible handheld device(s) 190 (i.e., the sanitization of the surface(s) 190S of the device is evident substantially coincident with viewing the device).

Referring again to FIG. 1, the shared fungible handheld device(s) 190 may be available for pick up by users, for example, at a docking station 300 (which may be coupled to the sanitizing station(s) 101 or separate and distinct from the sanitizing station(s) 101). The shared fungible handheld device(s) 190 that are ready for pick up are presented to the users in the docking station 300 so that presentation of the at least one of the shared fungible handheld device(s) 190 confirms the sanitized state of the shared fungible handheld device(s) 190 at a glance (i.e., upon a first instance of viewing).

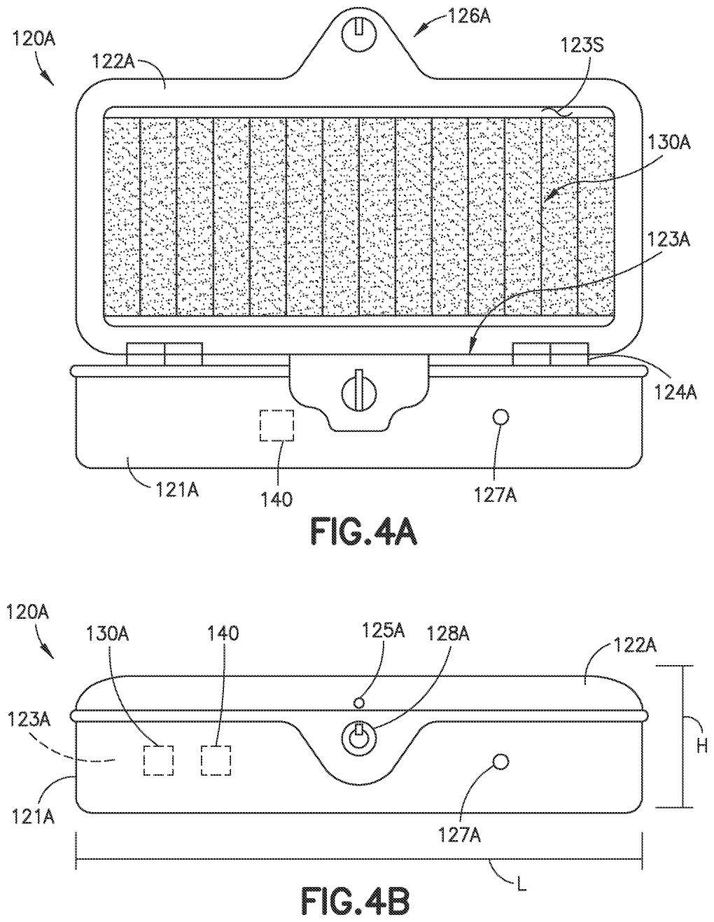

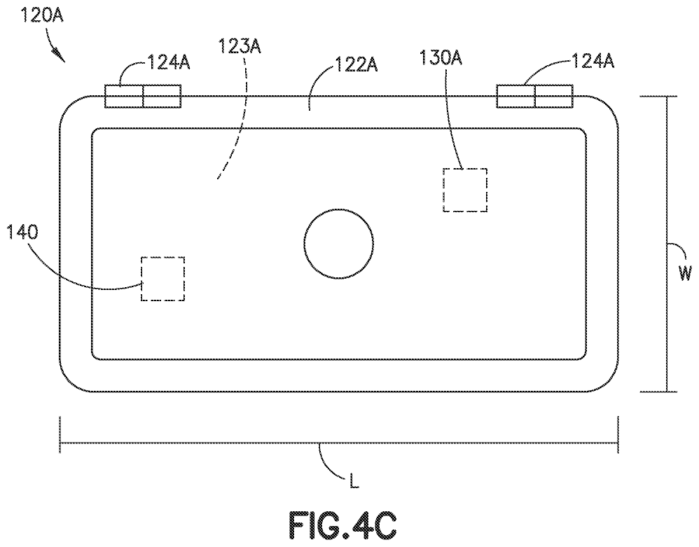

Referring now to FIGS. 1 and 3A-3C, 4A-4C, 5A-5C, and 6A-C, as noted above, the sanitizing station(s) 101 includes the transceiver 200 and at least one closure 120A-n configured to hold one or more of the shared fungible handheld device(s) 190 and the radiation source 130A-n enclosed therein. Each closure 120A-n includes a housing 121A-n and lid 122A-n which define an interior chamber 123A-n (FIG. 4A) of the closure 120A-n in which the one or more shared fungible handheld device(s) 190 are held for sanitization. For simplicity and ease of understanding, the remainder of the application will be discussed with respect to closure 120A, although what is disclosed is equally applicable to any one of the closures 120A-n.

The closure 120A may be manufactured of any suitable material that contains the UV radiation dispersed by the radiation source 130A disposed within the interior chamber 123A (i.e., blocking UV radiation from escaping the closure 120A). For example, the housing 121A and lid 122A may be manufactured from one or more of a metal, a polymer, glass, wood, ceramic, and/or any other suitable material for containing UV radiation. The housing 121A and lid 122A are shaped and sized (e.g., dimensioned) to receive and hold one or more of the shared fungible handheld device(s) 190 and the radiation source 130A disposed within the interior chamber 123A. In one aspect, the housing 121A and lid 122A may be dimensioned such that the interior chamber 123A defined by the housing 121A and the lid 122A is configured to hold one fungible handheld device 190; while in other aspects, the interior chamber 123A is configured to hold any suitable number of shared fungible handheld device(s) 190. For example, the housing 121A and the lid 122A have a length L of about 24 inches, a width W of about 15 inches, and a height of about 10 inches. In other aspects, the length L is greater than or less than 24 inches, the width W is greater than or less than 15 inches, and the height is greater than or less than 10 inches.

In one aspect, the interior chamber 123A includes one or more compartments or is configured to receive an inlay to change the configuration of the interior chamber 123A for receiving different shaped or sized devices. For example, in one aspect, the inlay is a tray shaped so as to hold a PDA or a cellular phone. In other aspects, the inlay is a rack configured to separate two devices 190 (e.g., stack one device above the other) within the interior chamber 123A.

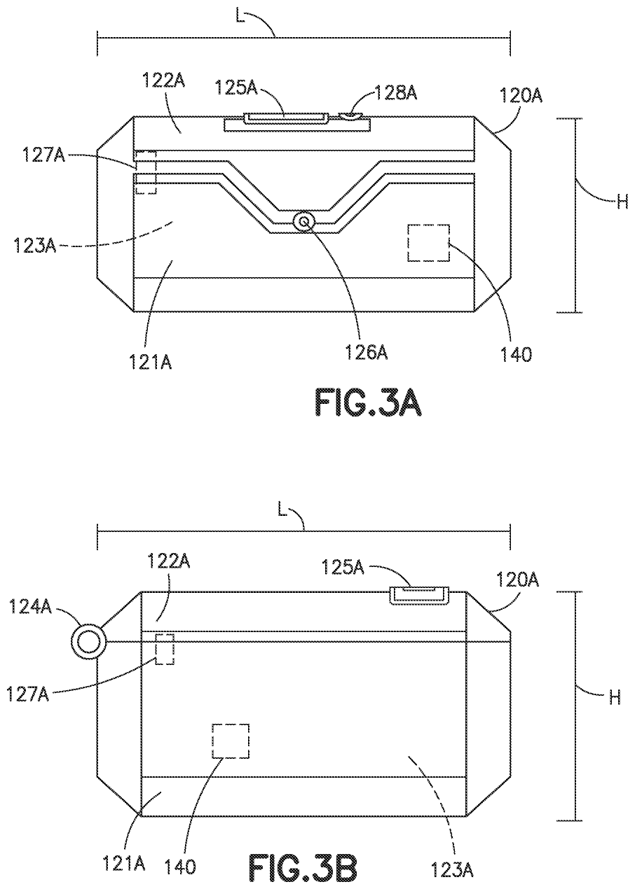

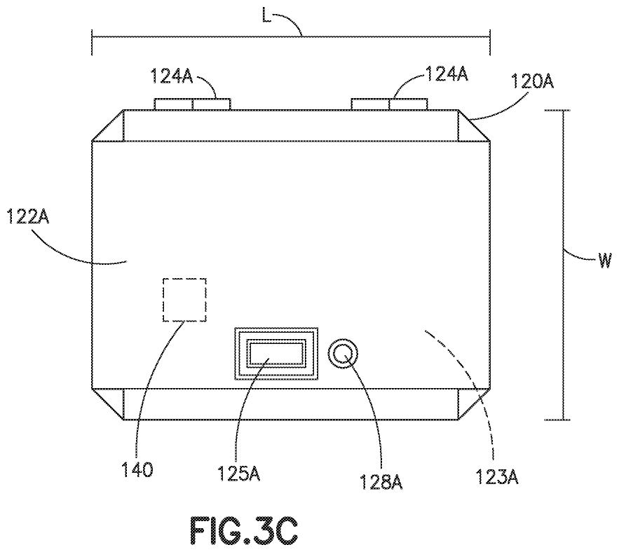

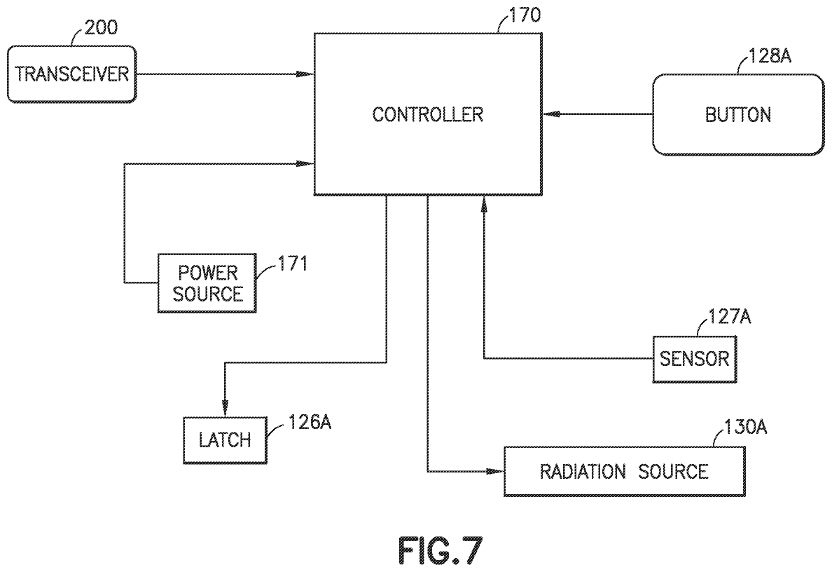

In one aspect, the lid 122A is connected to the housing 121A by hinge(s) 124A (FIG. 3B); however, in other aspects, the lid 122A is detachable (not hinged) from the housing 121A so as to be lifted off entirely. Opening or removal of the lid 122A (hinged or unhinged) may be manual or automatic. For example, the closure 120A may include a latch 126A (FIG. 3A) for unlocking and releasing the lid 122A from the housing 121A. In one aspect, the latch 126A is an electronic solenoid lock, a toggle latch, a cam/twist latch, or any other suitable latch. In one aspect, the closure 120A may further include an interlock. The interlock being provided so as to shut off or reduce power provided to the radiation source 130A from power source 171 (FIG. 7) when the latch 126A or lid 122A is unlocked/opened by a user. In another aspect, such as when the closure 120A has no latch, the interlock is provided so as to shut off or reduce power provided to the radiation source 130A from power source 171 when the lid 122A is opened by a user (e.g., “kill switch”). Providing the interlock may reduce exposure of the user to the UV radiation.

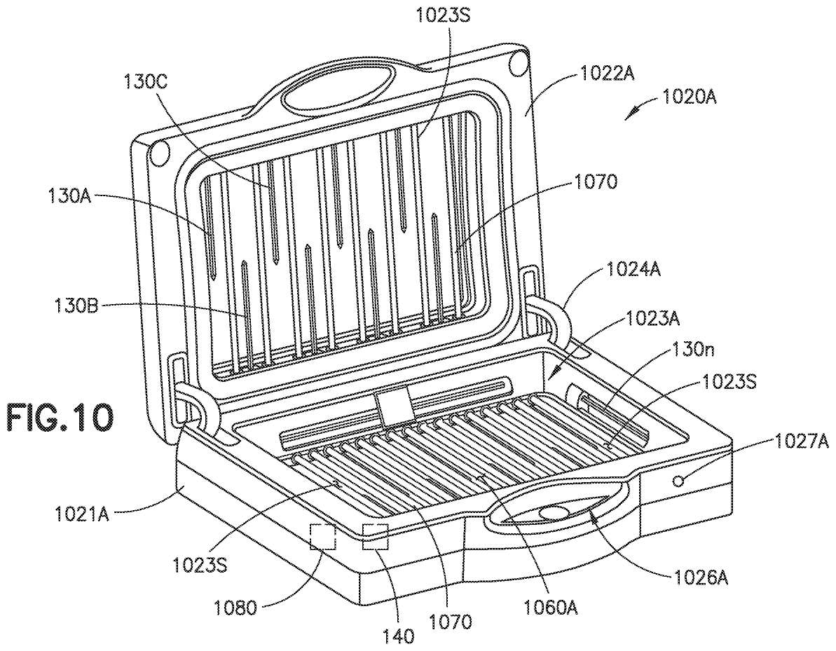

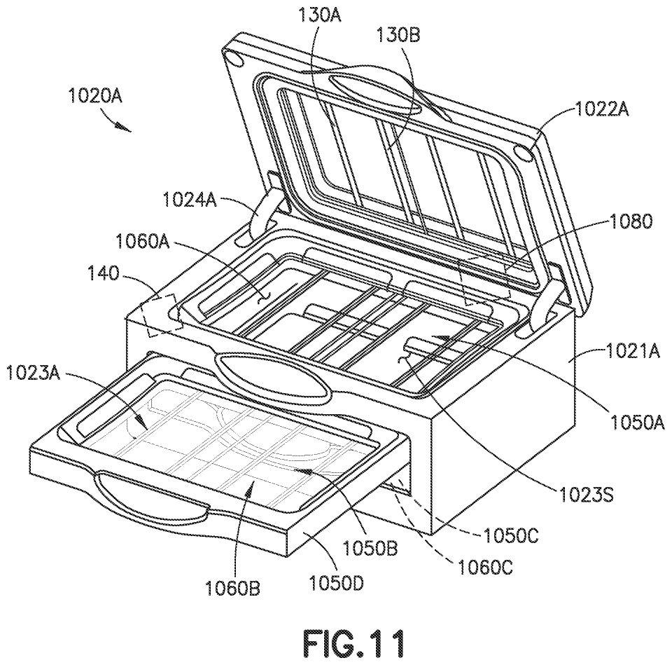

Still referring to FIGS. 1 and 3A-3C, 4A-4C, 5A-5C, and 6A-C, each closure 120A-n is configured such that the radiation source 130A-n, disposed within the closure 120A, is disposed to irradiate surface(s) 190S of the shared fungible handheld device(s) 190 held in the closure 120A. Irradiating the surface(s) 190S of the shared fungible handheld device(s) 190 with the UV radiation of the radiation source 130A-n effects sanitization of unsanitary surface(s) 190S of the shared fungible handheld device(s) 190 to the predetermined sanitization characteristic that determines the surface(s) 190S being sanitary. The radiation emitted by the radiation source 130A-n is of wavelengths anywhere from about 200 to about 300 nanometers, and generally about 266 nanometers; while in other aspects, the wavelengths are less than 200 nanometers or more than 300 nanometers. The dosage provided by the radiation source 130A-n is, as noted above, between about 40 mW/cm² and about 50 mW/cm², but may be less than 40 mW/cm² or more than 50 mW/cm². The electrical components needed to activate the radiation source 130A are disposed within the closure 120A (it should be noted that the power source 171 (FIG. 7) to the radiation source 130A may be derived from batteries, alternating current (A/C) source, or a combination of both, such as with a battery backup to the A/C source). In one aspect, the radiation source 130A may be activated via a remote network. Referring also to FIGS. 10 and 11, the closure may include one or more inverters 1080. Each radiation source 130A-n is configured to connect to a respective inverter 1080 to provide power to the radiation source 130A-n. In one aspect, multiple radiation sources 130A-n may connect to a common inverter 1080 (e.g., two radiation sources to one inverter). Utilizing inverters allows for the radiation source 130A-n to be plug and play such that when a radiation source 130A-n malfunctions or burns out, the single radiation source 130A-n can be replaced without having to replace all of the radiation source 130A-n.

In one aspect, an interior surface 123S (FIGS. 4A, 5A, and 6A) of the interior chamber 123A is configured to reflect UV radiation. The interior chamber 123A may be provided with at least one UV-reflective surface, so that the UV radiation is reflected, reducing absorption by the closure 120A-n walls. By reflecting the UV radiation within the closure 120A-n, the number of radiation sources 130A-n may be reduced. Here, the UV radiation is essentially trapped in a “light box,” thereby surrounding or bathing the shared fungible handheld device(s) 190 in UV radiation and improving the effectiveness of the sanitizing effect. One such way of providing reflective interior surfaces is to manufacture the housing 121A and lid 122A of the closure 120A from a reflective material such as a metal. In another aspect, the interior surfaces 123S may be provided with reflective surfaces, such as by inserting metallic elements, e.g., plates or foils, or coating the interior surfaces 123S with a UV reflective material, such as with aluminum alloys or any other suitable coatings.

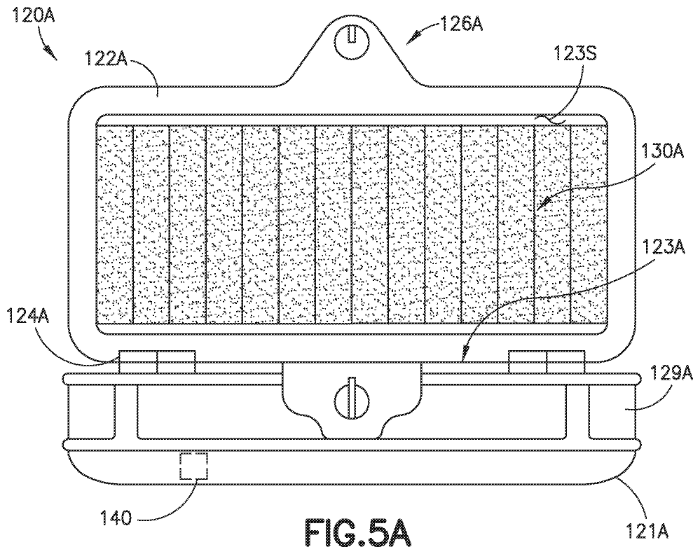

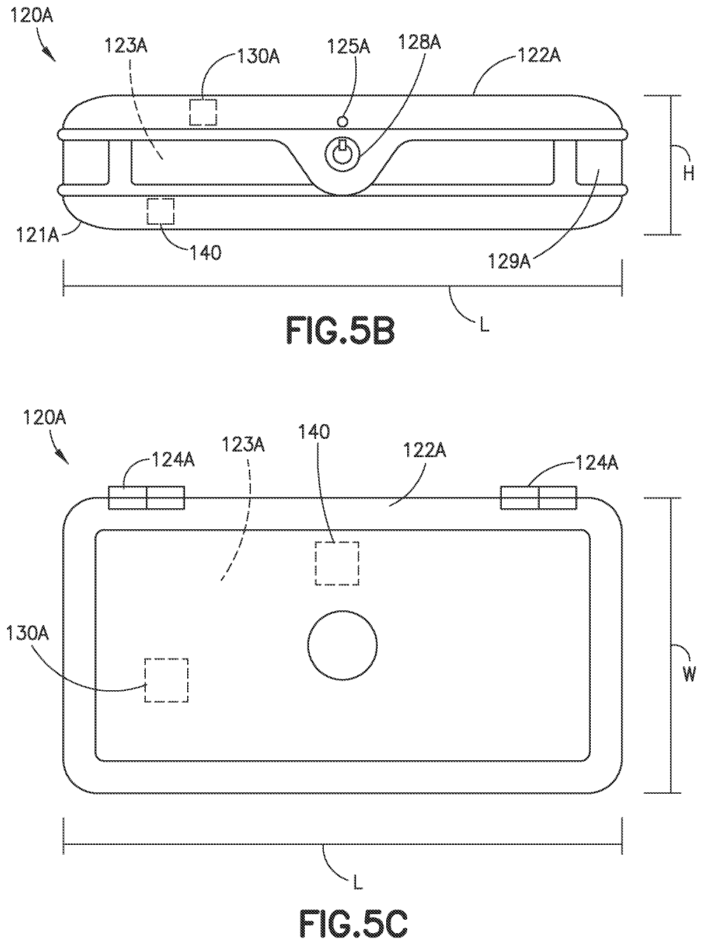

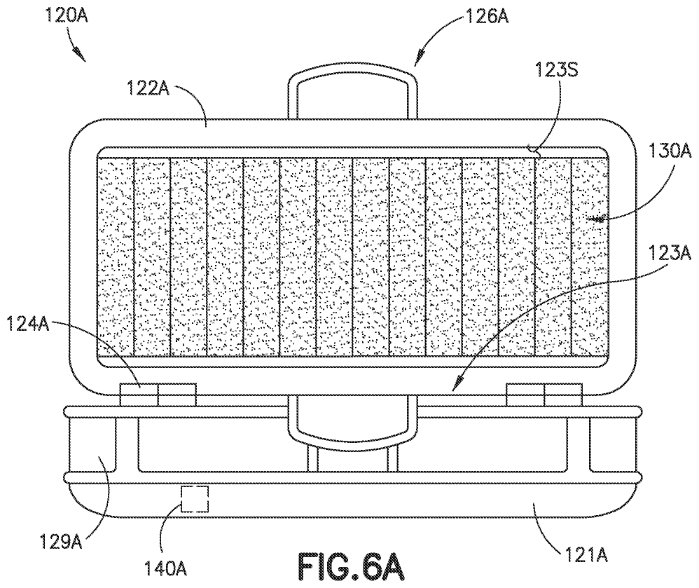

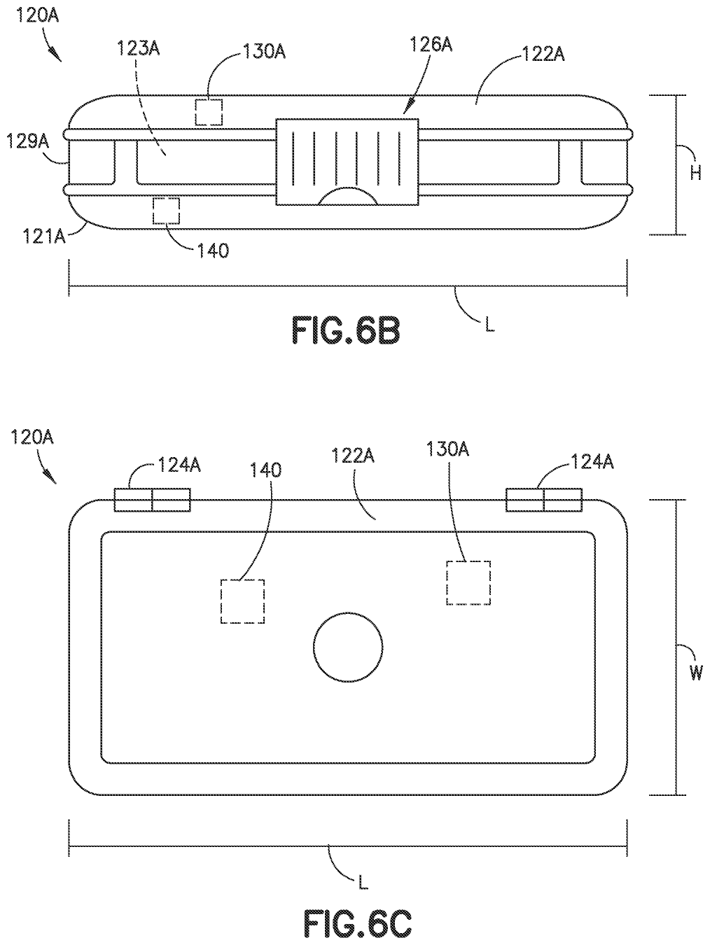

As seen best in FIGS. 5A-5C and 6A-6C, in one or more aspects, the housing 121A or lid 122A of the closure 120A may include a window 129A (FIG. 5A and 6A) for providing the user with visual egress into the interior chamber 123A. The window 129A extends completely or partially around the closure 120A. In one aspect, the window 129A is made of any suitable transparent or semi-transparent material such as glass, polycarbonate, polymethyl methacrylate (plexiglass). In one aspect, where the housing 121A and lid 122A are manufactured with a polymer, the polymer may be transparent in the visible spectrum, but block UV light (i.e., the polymers may include a UV blocking dye) to reduce the users exposure to the UV radiation.

Referring to FIGS. 1 and 3A-3C, 4A-4C, 5A-5C, 6A-C, and 7, in one aspect, the radiation source 130A disposed within the closure 120A may be initiated with a button 128A (FIG. 3A) provided on the closure 120A. The button 128A may be connected to the controller 170 such that when the button 128A is depressed by a user, the sanitization cycle (i.e., 15-30 second cleaning cycle or predetermined dosage cycle) is initiated (by, e.g., the controller 170). In another aspect, initiation of the sanitization cycle may commence upon closing of the lid 122A. For example, in this aspect, the housing 121A may include a trigger that is depressed by the lid 122A upon closing, where depressing of the trigger initiates the sanitization cycle. In yet another aspect, the closure 120A includes a sensor 127A (FIG. 3A), such as, an optical sensor or proximity sensor, which senses a presence of a user or the shared fungible handheld device(s) 190 to provide a touchless sanitizing station(s) 101 (i.e., the user does not have to touch any part of the closure 120A to open the closure 120A or activate the radiation source 130A and the sanitization cycle). For example, when the sensor 127A is a proximity sensor configured to sense a presence of the user, the sensor 127A, upon sensing motion, sends a signal to, e.g., the controller 170 to open the lid 122A for insertion of the shared fungible handheld device(s) 190. In one aspect, the sensor 127A is also utilized to initiate the sanitization cycle. For example, the sensor 127A senses the shared fungible handheld device(s) 190 being placed within the closure 120A to indicate to the lid 122A to close. With the lid 122A closed, the radiation source 130A is activated to start the sanitization cycle.

In one aspect, the closure 120A includes a UV sensor 140 configured to detect the presence of UV radiation or measure a dosage of UV radiation on the surface(s) 190S of the shared fungible handheld device(s) 190 (i.e., measure a predetermined sanitization characteristic). For example, the UV sensor 140 is a dosimeter or a photo sensor positioned within the closure 120A. The UV sensor 140 may be positioned in any suitable area of the interior at a predetermined distance from at least one of the UV tube (e.g., a distance of about 5 cm or a distance less than or greater than 5 cm). The controller 170 is communicably coupled to the UV sensor 140. Upon sensing, e.g., a predetermined dosage of radiation, the UV sensor 140 sends a signal to the controller 170 embodying the measure which is determinative of sanitization. The measure is sent to the transceiver 200 for communication to the shared fungible handheld device(s) 190 for indicating the shared fungible handheld device(s) 190 being in a sanitary state. In one aspect, the sensor 140 is configured so as to measure a dosage of radiation received by the unsanitary surface(s) of the at least one of multiple shared fungible handheld devices 190, via the radiation source 130A-n, and signal receipt of efficacious measured dose of radiation sanitization to each of the at least one of the multiple shared fungible handheld devices 190.

In one or more aspects, the closure 120A further includes status indicators 125A (FIG. 3A, 4A, 5A, and 6A) configured to convey, to the user, information related to the status of the closure 120A or one of the shared fungible handheld device(s) 190 enclosed therein. For example, such information may include the sanitary condition of each of the shared fungible handheld device(s) 190 held therein or any other suitable data. In one aspect, the status indicator 125A may be an LED light that illuminates upon, e.g., complete sanitization of one or more of the shared fungible handheld device(s) 190; in other aspects, the status indicator 125A is a display screen to indicate the aforementioned status(es).

Referring now to FIGS. 1A, 10, and 11, in another aspect, the sanitizing station(s) 101 of the sanitizing system 100 includes at least one closure 1020A-n configured to hold, enclosed therein, one or more of the shared fungible handheld or wearable device(s) 190 and a radiation source 130A-n (e.g., a UV lamp/tube, UV led, etc.). Each closure 1020A-n is substantially similar to closure 120A described above except for the differences noted herein. Each of the at least one closure 1020A-n of the sanitizing station(s) 101, with a respective radiation source 130A-n disposed therein (i.e., coupled to an interior surface 1023S of the closure 1020A), is configured so as to irradiate surface(s) 190S (FIG. 2) of a respective one of the shared fungible handheld or wearable device(s) 190 held within the respective closure 1020A. Irradiating the surface(s) 190S of the respective one of the shared fungible handheld or wearable device(s) 190 effects sanitization of unsanitary surface(s) to a predetermined sanitization characteristic (e.g., a characteristic determined by a predetermined dosage (e.g., between about 40 mW/cm2 and about 50 mW/cm2, but may be less than 40 mW/cm2 or more than 50 mW/cm2) of UV-C radiation effecting a determined disruption to potential pathogens), or any other suitable characteristic). In one aspect, sensor 140 is configured so as to measure a dosage of radiation received by the unsanitary surface(s) of the at least one of multiple shared fungible handheld devices 190, via the radiation source 130A-n, and signal receipt of efficacious measured dose of radiation sanitization to each of the at least one of the multiple shared fungible handheld devices 190. Each closure 1020A includes a housing 1021A and lid 1022A connected to the housing via hinge(s) 1024A which define an interior chamber 1023A of the closure 1020A in which the one or more shared fungible handheld device(s) 190 are held for sanitization. The housing 1021A may be sealed via latch 1026A and include a sensor 1027A similar to sensor 127A described above.

The housing 1021A and lid 1022A are shaped and sized (e.g., dimensioned) to receive and hold one or more of the shared fungible handheld device(s) 190 and the radiation sources 130A-n disposed within the interior chamber 1023A. In one aspect, the closure 1020A is a modular closure where the interior chamber 1023A comprises more than one separate compartments 1050A, 1050B, 1050C. In one aspect, compartment 1050A is substantially similar to the chamber 123A described above such that a portion of the housing 121A and the lid 122A define the dimensions of the compartment 1050A which is configured to hold at least one fungible handheld device 190. In one aspect, each compartment 1050A, 1050B, 1050C of the modular closure is self-contained and may be removed or inserted as a modular unit. The compartments 1050A, 1050B, 1050C may be plug and play (i.e., may include a controller configured to register the compartment 1050A, 1050B, 1050C upon insertion into the modular closure or deregister the compartment 1050A, 1050B, 1050C when removed (the controller reads/registers the compartment 1050A, 1050B, 1050C upon insertion/removal so as to allow irradiation of the surface(s) of the device, sensing and measuring dosage of the radiation and signaling receipt of efficacious measured dose of radiation sanitization). Each compartment 1050A, 1050B, 1050C has a fomite capacity max and may include an indication of sanitization for each fomite sanitized. The compartments 1050A, 1050B, 1050C may have a variably selective configuration with modifying adding/removing modules which add to or remove capacity to the compartments 1050A, 1050B, 1050C.

In one aspect, while compartments 1050B, 1050C are drawers 1050D which are separated from compartment 1050A (i.e., compartments without a lid, made to slide horizontally in and out of the housing 121A). Each of the compartments 1050A-1050C are configured to receive one or more different shaped or sized devices 190. In one aspect, the compartments 1050A-1050C are stacked one above the other and separated by a respective support deck 1060A-1060C configured to support the shared fungible handheld device(s) 190. In one aspect, the decks 1060A-1060C are comprised of a plurality of silica rods 1070 which are transparent to UV radiation to provide three hundred sixty-degree (360°) coverage of the surfaces of the shared fungible handheld device(s) 190 so as to sanitize each surface of the shared fungible handheld device(s) 190. The radiation source 130A-n may be placed below the deck 1060A so as to allow the surface of the shared fungible handheld device(s) 190 that is supported on the deck 1060A to be sanitized. (i.e., the UV tubes below the deck release UV-C light which passes through the silica deck and sanitizes the device surface). In another aspect, the decks 1060A-1060C are a shelf formed of silica so as to be transport transparent to UV radiation, allowing the UV radiation to travel between each compartment 1050A-1050C. The radiation sources 130A-n are positioned surrounding the shared fungible handheld device(s) 190 so that each surface of the shared fungible handheld device(s) 190 is exposed to UV radiation. Each radiation source 130A-n is positioned a predetermined distance from one another to optimize the UV radiation provided to the shared fungible handheld device(s) 190. In one aspect, each radiation sources 130A-n may be positioned so that each surface of the shared fungible handheld device(s) 190 is not more than 5 cm from a radiation source 130A-n. In one aspect, the predetermined distance may be more than 5 cm of may be less than 5 cm.

Referring to FIGS. 1 and 7, as noted above, the sanitizing system 100 may include the controller 170 connected to, e.g., the sanitizing station(s) 101 (it is noted than when a controller is not included in the system 100, the transceiver 200 is configured to transmit a signal to a separate beacon which transmits the indication to the respective shared fungible handheld devices 190). The controller 170 is configured to control power (provided from the power source 171) to the radiation source 130A (i.e., the controller turns on/off the radiation source 130A). When the controller 170 is included in the sanitizing system 100, the controller 170 is communicably coupled to the transceiver 200 and configured to generate the communication, communicated by the transceiver 200, to the shared fungible handheld device(s) 190 to indicate the shared fungible handheld device(s) 190 being sanitary. In one aspect, the controller 170 may also monitor inactivity of the sanitizing system 100 such that if the sanitizing system 100 is not utilized for a predetermined amount of time the controller 170 initiates a sanitizing cycle to sanitize the interior chamber 123A and/or any shared fungible handheld device(s) 190 located therein. In one aspect, the closure 120A, 1020A may include a system health monitoring function. For example, the controller 170 may monitor the current draw from the radiation source 130A-n. When one of the radiation sources 130A-n malfunction or cease to operate, the system health monitoring function of the controller 170 is configured to detect any discrepancy in the current draw and determine that at least one of the radiation sources 130A-n needs to be replaced.

As noted above, the sanitizing system 100 may be integrated with pre-existing systems (e.g., the system described in U.S. Pat. No. 7,856,368, the disclosure of which was previously incorporated herein by reference) having shared fungible handheld device(s) 190 docked at the docking stations 300. In one aspect, the closure 120A, 1020A-n may replace or function as the docking stations 300; while in other aspects, the docking stations 300 may be utilized separate and distinct from the closure 120A, 1020A-n. When the docking station 300 is separate and distinct from the closure 120A, 1020A-n, the docking stations 300 are configured so that presentation of the at least one of the shared fungible handheld device(s) 190 confirms the sanitized state of the shared fungible handheld device(s) 190 at a glance (i.e., upon a first instance of viewing). In one aspect, the transceiver 200 communicates the indication after opening of the closure 120A, 1020A-n post sanitization substantially coincident with placement of the shared fungible handheld device(s) 190 in the docking station 300. In one aspect, the sanitizing system 100 may only place (i.e., sanitarily by an administrator of the system 100) the shared fungible handheld device(s) 190 that are sanitized within the docking station so that a user may determine upon first glance that the shared fungible handheld device(s) 190 in the docking station 300 are sanitary. In one aspect, the docking station 300 is configured so as to charge the shared fungible handheld device(s) 190.

Referring to FIGS. 1 and 7, one or more of the shared fungible handheld device(s) 190, as described previously, are illustrated as being in one or more closures 120A-n, 1020A-n. As noted previously, the transceiver 200 of the sanitizing station(s) 101 may communicate with the shared fungible handheld device(s) 190 over the communication network 150. The communication network 150 may be an 802.11 network, Bluetooth, Global System for Mobile Communications (GSM), radio frequency (RF), Near Field Communication (NFC), infrared (IR) receiver, or any other suitable form of network. In other aspects, the communication network 150 may also be a wired network.

In one aspect, the communication link between the transceiver 200 and the shared fungible handheld device(s) 190 is established to a back end of a gateway 151 of the communication network 150 such that the gateway 151 is communicably link to the sanitizing station(s) 101 and the shared fungible handheld device(s) 190. The transceiver 200 is disposed to communicate the indication of the shared fungible handheld device(s) 190 being sanitary to the gateway 151, which indication is routed by the gateway 151 over the communication network 150 to one or more of the shared fungible handheld device(s) 190 in the closure 120A-n, 1020A-n that are in a sanitized state. In other aspects, any suitable means of communication or coupling may be available between the shared fungible handheld device(s) 190 and the sanitizing station(s) 101. In one aspect, the transceiver 200 is disposed to communicate to the gateway 151 of the communication network 150 an indication from the sanitizing station(s) 101 of the surface(s) 190S of the shared fungible handheld device(s) 190 in the closure 120A, 1020A-n being sanitary based on the predetermined sanitization characteristic being effected. The indication is routed by the gateway 151 over the communication network 150 to the shared fungible handheld device(s) 190 in the closure 120A, 1020A-n. In one aspect, the transceiver 200 communicates the indication upon/substantially coincident with, or proximately after opening of the closure 120A, 1020A-n post sanitization. In one aspect, the transceiver 200 communicates the indication upon/substantially coincident with, or proximately after termination of sanitization.

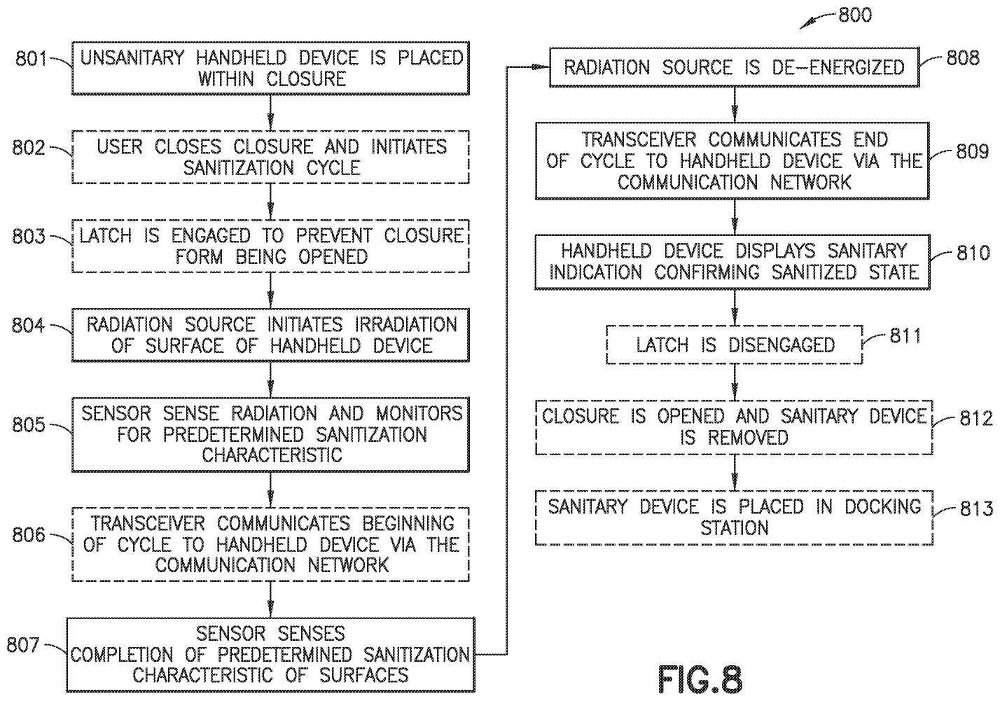

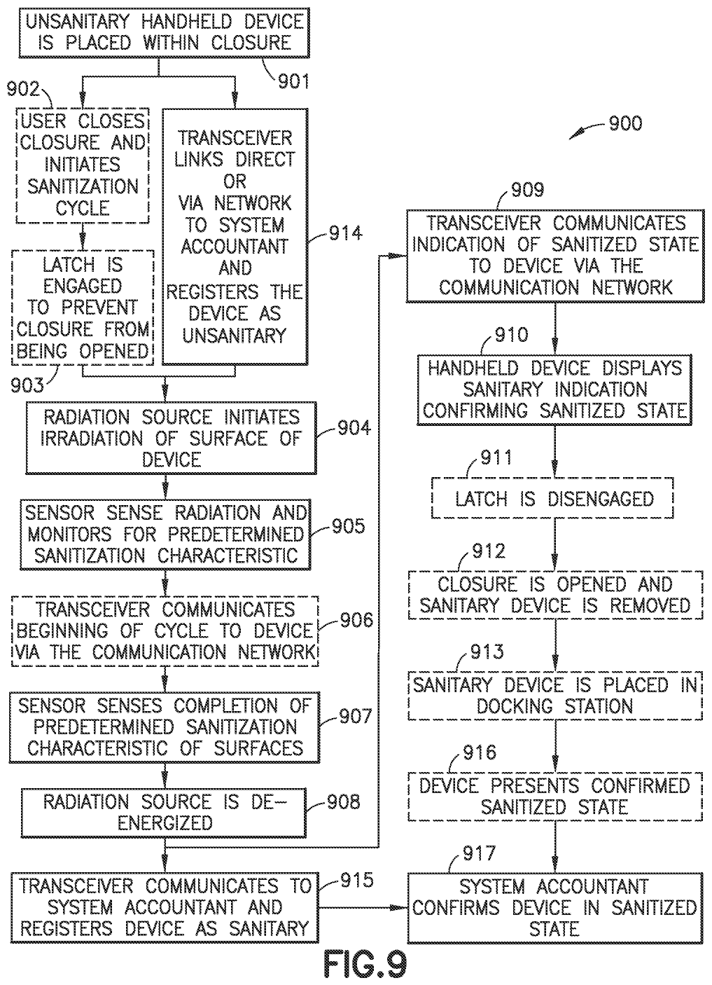

Referring to FIGS. 1, 8 and 9, a method 800, 900 for sanitizing shared fungible handheld device(s) 190 is illustrated. For example, a user may enter a facility having the sanitizing system 100 therein. The user locates one of the shared fungible handheld device(s) 190 in a closure 120A-n, 1020A-n or docking station 300 displaying an indication confirming a sanitized state. The user picks up the device 190 and utilizes the device throughout the facility thereby making the device 190 dirty or unsanitary. Once dirty, the device is sanitized where the user places the unsanitary device 190 into a closure 120A-n, 1020A-n (FIGS. 8 and 9, Blocks 801 and 901). The user closes the closure 120A-n, 1020A-n (or the closure 120A-n, 1020A-n automatically closes) and the sanitization cycle is initiated (FIGS. 8 and 9, Blocks 802 and 902). If the closure includes a latch 126A, the latch is engaged to prevent the closure from being reopened during the sanitization process (FIGS. 8 and 9, Blocks 803 and 903). In one aspect, upon receiving the unsanitary device 190 into a closure 120A-n, 1020A-n the transceiver 200 communicates a link, direct or via the communication network 150, to the system accountant 172 which registers the unsanitary device 190 in the system accountant as being unsanitary (FIG. 9, Block 914). When the sanitization cycle is initiated, the radiation source 130A-n disposed within the closure 120A-n, 1020A-n is activated or energized to irradiate the surface(s) of the device 190 enclosed in the closure 120A-n, 1020A-n (FIGS. 8 and 9, Blocks 804 and 904). A UV sensor 140 located within the closure 120A-n, 1020A-n senses the radiation from the radiation source 130A-n and measures the surface(s) 190s of the device 190 for a predetermined sanitization characteristic (FIGS. 8 and 9, Blocks 805 and 905). In one aspect, upon initialization of the sanitation cycle, the transceiver 200 may be configured to communicate to the device 190 via the network the sanitization cycle has begun (FIGS. 8 and 9, Blocks 806 and 906). When the sensor senses completion of the predetermined sanitization characteristic (FIGS. 8 and 9, Blocks 807 and 907), the radiation source 130A-n is deactivated or de-energized (FIGS. 8 and 9, Blocks 808 and 908). The transceiver 200 communicates to the handheld device via the communication network 150 an indication that the sanitization cycle is completed (FIGS. 8 and 9, Blocks 809 and 909). In one aspect, the transceiver 200 additionally communicates to the system accountant 172 to register the device 190 as being sanitary (FIG. 9, Block 915). The device 190 displays the indication that the sanitization cycle is completed which confirms to another user that the device 190 is in a sanitized state (FIGS. 8 and 9, Blocks 810 and 910). If the latch 126A is included on the closure 120A-n, 1020A-n, the latch is disengaged (FIGS. 8 and 9, Blocks 811 and 911). The closure 120A-n, 1020A-n is opened (manually by a user or automatically upon completion of the cycle) and the device 190 is removed from the closure 120A-n, 1020A-n (FIGS. 8 and 9, Blocks 812 and 912). In one aspect, the device 190 is sanitarily placed in the docking station 300 (FIGS. 8 and 9, Blocks 813 and 913), by an administrator of the system 100, such that presentation of the device 190 in the docking station 300 confirms the sanitized state of the device 190 at a glance. In one aspect, the device placed in the docking station 300 presents a confirmation of being in a sanitary state (FIG. 9, Block 916). In one aspect, with the device 190 being present in the docking station 300, the system accountant 172 confirms, based on the transceiver communication, whether the device 190 is in a sanitized state (FIG. 9, Block 917).

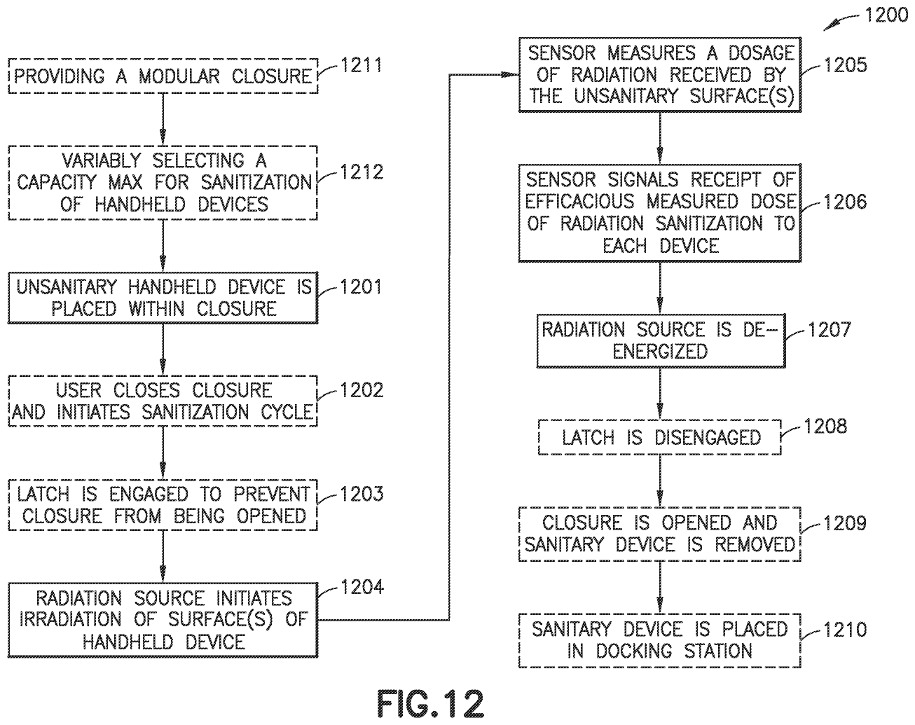

Referring to FIGS. 1A and 12, a method 1200 for sanitizing shared fungible handheld device(s) 190 is illustrated. A modular closure 1020A-n may be provided to sanitize shared fungible handheld device(s) 190 (FIG. 12, Block 1211). The modular closure 1020A-n may have a variably selective capacity max for sanitization of each of the shared fungible handheld device(s) 190 (FIG. 12, Block 1212). For example, a user may enter a facility having the sanitizing system 100 therein. The user locates one of the shared fungible handheld device(s) 190 in a closure 120A-n, 1020A-n or docking station 300 displaying an indication confirming a sanitized state. The user picks up the device 190 and utilizes the device throughout the facility thereby making the device 190 dirty or unsanitary. Once dirty, the device is sanitized where the user places the unsanitary device 190 into a closure 120A-n, 1020A-n (FIG. 12, Block 1201). The user closes the closure 120A-n, 1020A-n (or the closure 120A-n, 1020A-n automatically closes) and the sanitization cycle is initiated (FIG. 12, Block 1202). If the closure includes a latch 126A, the latch is engaged to prevent the closure from being reopened during the sanitization process (FIG. 12, Block 1203). When the sanitization cycle is initiated, the radiation source 130A-n disposed within the closure 120A-n, 1020A-n is activated or energized to irradiate the surface(s) of the device 190 enclosed in the closure 120A-n, 1020A-n (FIG. 12, Block 1204). A UV sensor 140 located within the closure 120A-n, 1020A-n senses and measures a dosage of the radiation from the radiation source 130A-n received by the surface(s) 190s of the device 190 (FIG. 12, Block 1205). Upon completion of sanitization, the sensor 140 signals receipt of efficacious measured dose of radiation sanitization to each device 190 (FIG. 12, Block 1206), the radiation source 130A-n is deactivated or de-energized (FIG. 12, Block 1207). If the latch 126A is included on the closure 120A-n, 1020A-n, the latch is disengaged (FIG. 12, Block 1208). The closure 120A-n, 1020A-n is opened (manually by a user or automatically upon completion of the cycle) and the device 190 is removed from the closure 120A-n, 1020A-n (FIG. 12, Block 1209). In one aspect, the device 190 is sanitarily placed in the docking station 300 (FIG. 12, Block 1210), by an administrator of the system 100, such that presentation of the device 190 in the docking station 300 confirms the sanitized state of the device 190 at a glance.

In accordance with one or more aspects of the disclosed embodiment a sanitizing system for sanitizing multiple shared fungible handheld devices is provided. The system includes a sanitizing station including a closure configured to hold enclosed therein at least one of the multiple shared fungible handheld devices, and a radiation source of UV-C radiation coupled to the closure and disposed to irradiate surface(s) of the at least one of multiple shared fungible handheld devices held in the closure effecting sanitization of unsanitary surface(s) of the at least one of the multiple shared fungible handheld devices to a predetermined sanitization characteristic that determines the surface(s) being sanitary, and a sensor configured so as to measure a dosage of radiation received by the unsanitary surface(s) of the at least one of multiple shared fungible handheld devices, via the radiation source, and signal receipt of efficacious measured dose of radiation sanitization to each of the at least one of the multiple shared fungible handheld devices.

In accordance with one or more aspects of the disclosed embodiment the system further includes a controller connected to the sanitizing station communicably coupled to the sensor, where the sensor signals the controller receipt of efficacious measured dose of radiation sanitization.

In accordance with one or more aspects of the disclosed embodiment the controller includes a system health monitoring function configured to monitor the radiation source for malfunction.

In accordance with one or more aspects of the disclosed embodiment the system further includes a docking station arranged for holding at least one of the multiple shared fungible handheld devices for pickup in a confirmed sanitized state, the sanitizing station being separate and distinct from the docking station.

In accordance with one or more aspects of the disclosed embodiment the docking station is configured so presentation of the at least one of the multiple shared fungible handheld devices confirms the sanitized state of the at least one of the multiple shared fungible handheld devices at a glance.

In accordance with one or more aspects of the disclosed embodiment one of the docking station or closure includes an indicator configured to indicate a dirty or clean state of the at least one of the multiple shared fungible handheld devices.

In accordance with one or more aspects of the disclosed embodiment the indicator displays an indication so that confirmation of sanitization of the device is evident substantially coincident with viewing the device.

In accordance with one or more aspects of the disclosed embodiment the closure includes side walls having at least one window for visual egress into the closure.

In accordance with one or more aspects of the disclosed embodiment the closure is modular.

In accordance with one or more aspects of the disclosed embodiment a method for sanitizing multiple shared fungible handheld devices is provided. The method includes providing a sanitizing station of a sanitizing system, the sanitizing station including a closure for holding enclosed therein at least one of the multiple shared fungible handheld devices, and a radiation source of UV-C radiation coupled to the closure and disposed for irradiating surface(s) of the at least one of multiple shared fungible handheld devices held in the closure effecting sanitization of unsanitary surface(s) of the at least one of the multiple shared fungible handheld devices to a predetermined sanitization characteristic that determines the surface(s) being sanitary, measuring, a dosage of radiation received by the unsanitary surface(s) of the at least one of multiple shared fungible handheld devices, via the radiation source, and signaling, with the sensor, receipt of efficacious measured dose of radiation sanitization to each of the at least one of the multiple shared fungible handheld devices.

In accordance with one or more aspects of the disclosed embodiment the sensor signals a controller connected to the sanitizing station communicably coupled to the sensor receipt of efficacious measured dose of radiation sanitization.

In accordance with one or more aspects of the disclosed embodiment the method further includes monitoring, with a system health monitoring function of the controller the radiation source for malfunction.

In accordance with one or more aspects of the disclosed embodiment the sanitizing system further includes a docking station arranged for holding at least one of the multiple shared fungible handheld devices for pickup in a confirmed sanitized state, the sanitizing station being separate and distinct from the docking station.

In accordance with one or more aspects of the disclosed embodiment the docking station is configured so presentation of the at least one of the multiple shared fungible handheld devices confirms the sanitized state of the at least one of the multiple shared fungible handheld devices at a glance.

15 In accordance with one or more aspects of the disclosed embodiment the method further includes indicating, with an indicator on one of the docking station or closure, a dirty or clean state of the at least one of the multiple shared fungible handheld devices.

In accordance with one or more aspects of the disclosed embodiment the indicator displays an indication so that confirmation of sanitization of the device is evident substantially coincident with viewing the device.

17 In accordance with one or more aspects of the disclosed embodiment the closure includes side walls having at least one window for visual egress into the closure.

In accordance with one or more aspects of the disclosed embodiment the closure is modular.

In accordance with one or more aspects of the disclosed embodiment the modular closure has a variably selective capacity max.

In accordance with one or more aspects of the disclosed embodiment a sanitizing system for sanitizing multiple shared fungible handheld or wearable devices communicably connected to a wireless communication network is provided. The system includes a sanitizing station including a closure configured to hold enclosed therein more than one of the multiple shared fungible handheld or wearable devices, and a radiation source of UV-C radiation coupled to the closure and disposed to irradiate surface(s) of the multiple shared fungible handheld or wearable devices held in the closure effecting sanitization of unsanitary surface(s) of the multiple shared fungible handheld or wearable devices to a predetermined sanitization characteristic that determines the surface(s) being sanitary, and a transceiver connected to the closure and configured to communicate via a communication link, established by the transceiver, via the network, or direct to at least one of the multiple shared fungible handheld or wearable devices in the closure an indication from the sanitizing station, based on the predetermined sanitization characteristic being effected, of the surface(s) of the at least one of the multiple shared fungible handheld or wearable devices in the closure being sanitary.

In accordance with one or more aspects of the disclosed embodiment the sanitizing station communicates the indication of the surface(s) being sanitary via the link.

In accordance with one or more aspects of the disclosed embodiment the indication received by the at least one of the multiple shared fungible handheld or wearable devices in the closure is disposed by the at least one of the multiple shared fungible handheld or wearable devices in the closure so as to present confirmation of sanitization of the at least one of the multiple shared fungible handheld or wearable devices in the closure.

In accordance with one or more aspects of the disclosed embodiment the at least one of the multiple shared fungible handheld or wearable devices in the closure displays the indication so that confirmation of sanitization of the at least one of the multiple shared fungible handheld or wearable devices in the closure is evident from the surface of the at least one of the multiple shared fungible handheld or wearable devices in the closure substantially coincident with viewing the device.

In accordance with one or more aspects of the disclosed embodiment the communication link is established to a back end of a gateway of the wireless communication network so as to communicably link the gateway and sanitizing station, and the transceiver is disposed to communicate the indication to the gateway, which indication is routed by the gateway over the network to the at least one of the multiple shared fungible handheld or wearable devices in the closure.

In accordance with one or more aspects of the disclosed embodiment the system further includes a controller connected to the sanitizing station communicably coupled to a sensor disposed on the closure configured to sense a measure of the predetermined sanitization characteristic and send a signal to the controller embodying the measure sensed by the sensor, which measure is determinative of the predetermined sanitization characteristic.

In accordance with one or more aspects of the disclosed embodiment the controller is communicably coupled to the transceiver and configured to generate a communication communicated by the transceiver with the indication of the handheld or wearable device being sanitary.

In accordance with one or more aspects of the disclosed embodiment the system further includes a docking station arranged for holding at least one of the multiple shared fungible handheld or wearable devices for pickup in a confirmed sanitized state, the sanitizing station being separate and distinct from the docking station.

In accordance with one or more aspects of the disclosed embodiment the docking station is configured so presentation of the at least one of the multiple shared fungible handheld or wearable devices confirms the sanitized state of the at least one of the multiple shared fungible handheld or wearable devices at a glance

In accordance with one or more aspects of the disclosed embodiment the transceiver communicates the indication after opening of the closure post sanitization substantially coincident with placement of the at least one of the multiple shared fungible handheld or wearable devices in the docking station.

In accordance with one or more aspects of the disclosed embodiment the at least one of the multiple shared fungible handheld or wearable devices held at the docking station provides the indication forming the presentation by the docking station that confirms the device held is in the sanitized state.

In accordance with one or more aspects of the disclosed embodiment the transceiver communicates the indication upon/substantially coincident with, or proximately after opening of the closure post sanitization.

In accordance with one or more aspects of the disclosed embodiment the transceiver communicates the indication upon/substantially coincident with, or proximately after termination of sanitization.

In accordance with one or more aspects of the disclosed embodiment a sanitizing system for sanitizing multiple shared fungible handheld or wearable devices communicably connected to a wireless communication network. The system includes a sanitizing station including a closure configured to hold enclosed therein more than one of the multiple shared fungible handheld or wearable devices, and a radiation source of UV-C radiation coupled to the closure and disposed to irradiate surface(s) of the more than one of the multiple shared fungible handheld or wearable devices held in the closure effecting sanitization of unsanitary surface(s) of the more than one of the multiple shared fungible handheld or wearable devices to a predetermined sanitization characteristic that determines the surface(s) being sanitary, a transceiver connected to the closure and configured to communicate to a gateway of the wireless communication network so as to communicably link the gateway and sanitizing station, and the transceiver is disposed to communicate to the gateway an indication from the sanitizing station, based on the predetermined sanitization characteristic being effected, of the surface(s) of the more than one of the multiple shared fungible handheld or wearable devices in the closure being sanitary, which indication is routed by the gateway over the network to the more than one of the multiple shared fungible handheld or wearable devices in the closure.

In accordance with one or more aspects of the disclosed embodiment the sanitizing station communicates the indication of the surface(s) being sanitary via the link.

In accordance with one or more aspects of the disclosed embodiment the indication received by the more than one of the multiple shared fungible handheld or wearable devices in the closure is disposed by the more than one of the multiple shared fungible handheld or wearable devices in the closure so as to present confirmation of sanitization of the more than one of the multiple shared fungible handheld or wearable devices in the closure.

In accordance with one or more aspects of the disclosed embodiment the more than one of the multiple shared fungible handheld or wearable devices in the closure displays the indication so that confirmation of sanitization of the more than one of the multiple shared fungible handheld or wearable devices in the closure is evident from the surface of the more than one of the multiple shared fungible handheld or wearable devices in the closure substantially coincident with viewing the device.

In accordance with one or more aspects of the disclosed embodiment the communication link is established to a back end of the gateway of the wireless communication network so as to communicably link the gateway and sanitizing station.

In accordance with one or more aspects of the disclosed embodiment the system further includes a controller connected to the sanitizing station communicably coupled to a sensor disposed on the closure configured to sense a measure of the predetermined sanitization characteristic and send a signal to the controller embodying the measure sensed by the sensor, which measure is determinative of the predetermined sanitization characteristic.

In accordance with one or more aspects of the disclosed embodiment the controller is communicably coupled to the transceiver and configured to generate a communication communicated by the transceiver with the indication of the handheld or wearable device being sanitary.

In accordance with one or more aspects of the disclosed embodiment the system further includes a docking station arranged for holding the multiple shared fungible handheld or wearable devices for pickup in a confirmed sanitized state, the sanitizing station being separate and distinct from the docking station.

In accordance with one or more aspects of the disclosed embodiment the docking station is configured so presentation of the multiple shared fungible handheld or wearable devices confirms the sanitized state of the multiple shared fungible handheld or wearable devices at a glance

In accordance with one or more aspects of the disclosed embodiment the transceiver communicates the indication after opening of the closure post sanitization substantially coincident with placement of the multiple shared fungible handheld or wearable devices in the docking station.

In accordance with one or more aspects of the disclosed embodiment the multiple shared fungible handheld or wearable devices held at the docking station provides the indication forming the presentation by the docking station that confirms the device held is in the sanitized state.

In accordance with one or more aspects of the disclosed embodiment the transceiver communicates the indication upon/substantially coincident with, or proximately after opening of the closure post sanitization.

In accordance with one or more aspects of the disclosed embodiment the transceiver communicates the indication upon/substantially coincident with, or proximately after termination of sanitization.

In accordance with one or more aspects of the disclosed embodiment a sanitizing system for sanitizing multiple shared fungible handheld or wearable devices of a wireless communication network. The system includes a docking station arranged for holding at least one shared fungible handheld or wearable device for pickup in a confirmed sanitized state, the station being configured so presentation of the at least one shared fungible handheld or wearable device confirms the sanitized state of the at least one shared fungible handheld or wearable device at a glance, a sanitizing station including a closure configured to hold enclosed therein the at least one handheld or wearable device, and a radiation source of UV-C radiation coupled to the closure and disposed to irradiate surface(s) of the handheld or wearable device held in the closure effecting sanitization of unsanitary surface(s) of the handheld or wearable device to a predetermined sanitization characteristic that determines the surface(s) being sanitary, and a transceiver connected to the closure and configured to communicate via a communication link, formed by the transceiver, via the network, or direct to the at least one handheld or wearable device in the closure an indication from the sanitizing station, based on the predetermined sanitization characteristic being effected, of the surface(s) of the handheld or wearable devices in the closure being sanitary, wherein the indication received by the handheld or wearable device confirms the surfaces of the handheld or wearable device being sanitized so that the docking station holding the handheld or wearable device presents the indication confirming the sanitized state.

In accordance with one or more aspects of the disclosed embodiment the sanitizing station is separate and distinct from the docking station.

In accordance with one or more aspects of the disclosed embodiment the transceiver communicates the indication upon/substantially coincident with, or proximately after opening of the closure post sanitization.

In accordance with one or more aspects of the disclosed embodiment the transceiver communicates the indication upon/substantially coincident with, or proximately after termination of sanitization.

In accordance with one or more aspects of the disclosed embodiment the transceiver communicates the indication after opening of the closure post sanitization substantially coincident with placement of the handheld or wearable device in the docking station.

In accordance with one or more aspects of the disclosed embodiment the handheld or wearable device held at the docking station provides the indication forming the presentation by the docking station that confirms the device held is in the sanitized state.

In accordance with one or more aspects of the disclosed embodiment the sanitizing station communicates the indication of the surface(s) being sanitary via the link.

In accordance with one or more aspects of the disclosed embodiment the indication received by the handheld or wearable device is disposed by the handheld or wearable device so as to present confirmation of sanitization of the handheld or wearable device.

In accordance with one or more aspects of the disclosed embodiment the handheld or wearable device displays the indication so that confirmation of sanitization of the device is evident from the surface of the device substantially coincident with viewing the device.

In accordance with one or more aspects of the disclosed embodiment the communication link is established to a back end of a gateway of the wireless communication network so as to communicably link the gateway and sanitizing station, and the transceiver is disposed to communicate the indication to the gateway, which indication is routed by the gateway over the network to the handheld or wearable devices in the closure.

In accordance with one or more aspects of the disclosed embodiment the system further includes a controller connected to the sanitizing station communicably coupled to a sensor disposed on the closure configured to sense a measure of the predetermined sanitization characteristic and send a signal to the controller embodying the measure sensed by the sensor, which measure is determinative of the predetermined sanitization characteristic.

In accordance with one or more aspects of the disclosed embodiment the controller is communicably coupled to the transceiver and configured to generate a communication communicated by the transceiver with the indication of the handheld or wearable device being sanitary.

In accordance with one or more aspects of the disclosed embodiment a system for sanitizing at least one handheld or wearable device is provided. The system including a housing defining an enclosure configured to receive the at least one handheld or wearable device, at least one ultraviolet radiation source coupled to the housing, the at least one ultraviolet radiation source configured to emit sanitizing UV radiation within the enclosure,

a controller operably coupled to the at least one ultraviolet radiation source, the controller configured to supply power to the at least one ultraviolet radiation source so as to effect sanitation of surfaces of the at least one handheld or wearable device disposed within the enclosure to a predetermined sanitization characteristic that determines the surfaces being sanitary, and a gateway configured so as to communicably connect the controller and the at least one handheld or wearable device so that upon effecting sanitization of the surfaces of the at least one handheld or wearable device, the controller communicates to the at least one handheld or wearable device to display a sanitized indication confirming sanitization of the at least one handheld or wearable device based on the predetermined sanitization characteristic that determines the surfaces being sanitary.

In accordance with one or more aspects of the disclosed embodiment the frequency wavelength of the UV radiation of the at least one ultraviolet radiation source is about 266 nano.

In accordance with one or more aspects of the disclosed embodiment the housing includes a lid and a base.

In accordance with one or more aspects of the disclosed embodiment the lid includes latch configured to lock the housing.

In accordance with one or more aspects of the disclosed embodiment the latch is an electronic latch.

In accordance with one or more aspects of the disclosed embodiment the housing includes an indicator configured to indicate a dirty or clean state of the at least one handheld or wearable device (e.g., an led light).

In accordance with one or more aspects of the disclosed embodiment the housing includes side walls having at least one window for visual egress into the housing.

In accordance with one or more aspects of the disclosed embodiment the window is comprised of a transparent or semi-transparent material.

In accordance with one or more aspects of the disclosed embodiment the system further includes sensors for automatically opening/closing the housing.

In accordance with one or more aspects of the disclosed embodiment the controller is configured to determine a length of time since last sanitization of each handheld or wearable device.

In accordance with one or more aspects of the disclosed embodiment the system further includes an inlay configured for insertion into the housing changing a configuration of the housing. It should be understood that the foregoing description is only illustrative of the aspects of the disclosed embodiment and that the aspects of the disclosed embodiment can be used individually or in any suitable combination thereof. Various alternatives and modifications can be devised by those skilled in the art without departing from the aspects of the disclosed embodiment. Accordingly, the aspects of the disclosed embodiment are intended to embrace all such alternatives, modifications and variances.

What is claimed is:

1. A sanitizing system for sanitizing multiple shared fungible handheld devices, the system comprising:
a sanitizing station including a closure configured to hold enclosed therein at least one of the multiple shared fungible handheld devices, and a radiation source of UV-C radiation coupled to the closure and disposed to irradiate surface(s) of the at least one of multiple shared fungible handheld devices held in the closure effecting sanitization of unsanitary surface(s) of the at least one of the multiple shared fungible handheld devices to a predetermined sanitization characteristic that determines the surface(s) being sanitary; and
a sensor configured so as to measure a dosage of radiation received by the unsanitary surface(s) of the at least one of multiple shared fungible handheld devices, via the radiation source, and signal receipt of efficacious measured dose of radiation sanitization to each of the at least one of the multiple shared fungible handheld devices.

2. The sanitizing system of claim 1, further comprising a controller connected to the sanitizing station communicably coupled to the sensor, where the sensor signals the controller receipt of efficacious measured dose of radiation sanitization.

3. The sanitizing system of claim 2, wherein the controller includes a system health monitoring function configured to monitor the radiation source for malfunction.

4. The sanitizing system of claim 1, further comprising a docking station arranged for holding at least one of the multiple shared fungible handheld devices for pickup in a confirmed sanitized state, the sanitizing station being separate and distinct from the docking station.

5. The sanitizing system of claim 4, wherein the docking station is configured so presentation of the at least one of the multiple shared fungible handheld devices confirms the sanitized state of the at least one of the multiple shared fungible handheld devices at a glance.

6. The sanitizing system of claim 1, wherein one of the docking station or closure includes an indicator configured to indicate a dirty or clean state of the at least one of the multiple shared fungible handheld devices.

7. The sanitizing system of claim 1, wherein the indicator displays an indication so that confirmation of sanitization of the device is evident substantially coincident with viewing the device.

8. The sanitizing system of claim 1, wherein the closure includes side walls having at least one window for visual egress into the closure.

9. The sanitizing system of claim 1, wherein the closure is modular.

10. A method for sanitizing multiple shared fungible handheld devices, the method comprising:
providing a sanitizing station of a sanitizing system, the sanitizing station including a closure for holding enclosed therein at least one of the multiple shared fungible handheld devices, and a radiation source of UV-C radiation coupled to the closure and disposed for irradiating surface(s) of the at least one of multiple shared fungible handheld devices held in the closure effecting sanitization of unsanitary surface(s) of the at least one of the multiple shared fungible handheld devices to a predetermined sanitization characteristic that determines the surface(s) being sanitary;
measuring, a dosage of radiation received by the unsanitary surface(s) of the at least one of multiple shared fungible handheld devices, via the radiation source; and
signaling, with the sensor, receipt of efficacious measured dose of radiation sanitization to each of the at least one of the multiple shared fungible handheld devices.

11. The method of claim 10, wherein the sensor signals a controller connected to the sanitizing station communicably coupled to the sensor receipt of efficacious measured dose of radiation sanitization.

12. The method of claim 11, further comprising monitoring, with a system health monitoring function of the controller the radiation source for malfunction.

13. The method of claim 10, wherein the sanitizing system further includes a docking station arranged for holding at least one of the multiple shared fungible handheld devices for pickup in a confirmed sanitized state, the sanitizing station being separate and distinct from the docking station.

14. The method of claim 13, wherein the docking station is configured so presentation of the at least one of the multiple shared fungible handheld devices confirms the sanitized state of the at least one of the multiple shared fungible handheld devices at a glance.

15. The method of claim 10, further comprising indicating, with an indicator on one of the docking station or closure, a dirty or clean state of the at least one of the multiple shared fungible handheld devices.

16. The method of claim 10, wherein the indicator displays an indication so that confirmation of sanitization of the device is evident substantially coincident with viewing the device.

17. The method of claim 10, wherein the closure includes side walls having at least one window for visual egress into the closure.

18. The method of claim 10, wherein the closure is modular.

19. A sanitizing system for sanitizing multiple shared fungible handheld devices communicably connected to a wireless communication network, the system comprising:
a sanitizing station including a closure configured to hold enclosed therein more than one of the multiple shared fungible handheld devices, and a radiation source of UV-C radiation coupled to the closure and disposed to irradiate surface(s) of the multiple shared fungible handheld devices held in the closure effecting sanitization of unsanitary surface(s) of the multiple shared fungible handheld devices to a predetermined sanitization characteristic that determines the surface(s) being sanitary; and
a transceiver connected to the closure and configured to communicate via a communication link, established by the transceiver, via the network, or direct to at least one of the multiple shared fungible handheld devices in the closure an indication from the sanitizing station, based on the predetermined sanitization characteristic being effected, of the surface(s) of the at least one of the multiple shared fungible handheld devices in the closure being sanitary.

20. The sanitizing system of claim 19, wherein the indication received by the at least one of the multiple shared fungible handheld devices in the closure is disposed by the at least one of the multiple shared fungible handheld devices in the closure so as to present confirmation of sanitization of the at least one of the multiple shared fungible handheld devices in the closure.
